(19) United States
(12) Patent Application Publication
(10) Pub. No.: US 2016/0039885 A1
(43) Pub. Date: Feb. 11, 2016

(54) RECOMBINANT PRODUCTION OF HIV-1 ENVELOPE GLYCOPROTEINS

(71) Applicant: DUKE UNIVERSITY, Durham, NC (US)

(72) Inventors: Barton F. Haynes, Durham, NC (US); Hua-Xin Liao, Durham, NC (US); Jae-sung Yu, Durham, NC (US)

(21) Appl. No.: 14/781,881

(22) PCT Filed: Apr. 1, 2014
(86) PCT No.: PCT/US2014/032497

§ 371 Date: Oct. 1, 2015

Related U.S. Application Data
(60) Provisional application No. 61/807,644, filed on Apr. 2, 2013.

(51) Int. Cl. C07K 14/005; C12N 7/00

(52) U.S. Cl. CPC C07K 14/005; C12N 7/00; C12N 2740/16134; C12N 2740/16122

(57) ABSTRACT

The present invention relates, in general, to HIV-1 and, in particular, to methods of producing HIV-1 envelope (Env) proteins, and subunits thereof, and to constructs suitable for use in such methods.

The present application claims benefit of U.S. Provisional Application No. 61/807,644 filed Apr. 2, 2013, the entire contents of which is incorporated herein by reference.

This invention was made with government support under Grant No. UM1 AI 1000645 awarded by the National Institutes of Health. The government has certain rights in the invention.

### TECHNICAL FIELD

The present invention relates, in general, to HIV-1 and, in particular, to methods of producing HIV-1 envelope (Env) proteins, and subunits thereof, and to constructs suitable for use in such methods.

### BACKGROUND

For the development of an effective vaccine against human immunodeficiency virus type 1 (HIV-1), recombinant expression of the HIV-1 Env, or Env subunit, as an immunogen will likely be required. In this regard, the induction of HIV-1 broadly neutralizing antibodies (BnAbs) is a key goal of HIV-1 vaccine development. HIV-1 glycoprotein is the target for development of a neutralizing antibody-based vaccine.

Recombinant HIV-1 glycoproteins in the form of gp 120 and gp 140, as well as other forms, have been under development as vaccine immunogens. For use in human clinical trials, recombinant Env glycoproteins need to be produced in mammalian cell lines that fulfill regulatory requirements for ultimate testing in humans. The most commonly used such cell line is the Chinese hamster ovary cell line (CHO) (Kim et al, Appl. Microbiol. Biotechnol. 93:917-930 (2012)).

HIV-1 glycoproteins can be efficiently produced as intact proteins in Human Embryonic Kidney 293 cells (HEK 293). However, HEK293 cells are not currently approved for production of clinical grade material.

CHO cells can be easily adapted to growth in serum-free media and can produce glycosylated recombinant proteins (Sheeley et al, Anal. Biochem. 247:102-110 (1997), Werner et al, Arneimittelforschung 48:870-880 (1998)). A disadvantage of the expression system in CHO cells is low productivity (Kim et al, Appl. Microbiol. Biotechnol. 93:917-930 (2012)). However, this disadvantage can by overcome by use of a gene amplification procedure involving the use of dihydrofolate reductase (DHFR)-deficient CHO cells (Reff, Curr. Opin. Biotechnol. 4:573-576 (1993), Trill et al, Curr. Opin. Biotechnol. 6:553-560 (1995)). DHFR activity is essential for DNA synthesis—DHFR-deficient CHO cells require hypoxanthine and thymidine (HT) for growth. If methotrexate (MTX) is present in the medium, it is converted to a high molecular weight polyglutamate metabolite that binds to and inhibits DHFR activity, leading to cell death. However, DHFR-deficient CHO cells transfected with an expression vector containing the DHFR gene compensate by increasing the DHFR copy number in the genome to overcome inhibition by MTX (Kaufman et al, Mol. Cell Biol. 6:1750-1759 (1985), Tanaka et al, Proc. Natl. Acad. Sci. USA 99:8772-8777 (2002)). Because the amplification unit is much larger (100 to 3,000 kb) than the size of the DHFR gene, a specific gene of interest, which is co-linked to the DHFR gene in the expression vector or that resides adjacently in the host chromosome, is co-amplified (Kaufman et al, Mol. Cell Biol. 3:699-711 (1983)). Since the HIV-1 envelope (env) gene is integrated into the same genetic locus as DHFR gene, the env gene is amplified as well.

The present invention results, at least in part, from studies relating to the development of stable cell lines that produce HIV-1 Env proteins. These studies involved the development of a eukaryotic expression vector, pHV100009, which has multiple cloning sites from the pcDNA3.1.(+)/Hygro vector and is a derivative of pOptiVEC-TOPO vector (Invitrogen, Carlsbad, Calif.). The pHV100009 expression vector includes the DHFR gene and several unique cloning sites (e.g., NheI, EcoRI, PstI, EcoRV, and XhoI). HIV-1 gp120 and gp140 genes can be cloned, for example, into NheI and Xbal sites in the pHV100009 vector. The invention provides an approach to producing HIV-1 Env proteins in CHO cells that avoids cleavage often associated with expression of these proteins in such cells

### SUMMARY OF THE INVENTION

The present invention relates, in general, to HIV-1. More specifically, the invention relates to recombinant methods of producing HIV-1 Env proteins, and subunits thereof. The invention also relates to constructs suitable for use in such methods.

Objects and advantages of the present invention will be clear from the description that follows.

### BRIEF DESCRIPTION OF THE DRAWINGS

FIG. 1. Western blot analysis of HIV-1 gp120 Env proteins expressed in DHFR-deficient CHO cells by stable transfection. The HIV-1 gp120 Env proteins were fractionated on 4-12% gradient SDS-PAGE under reducing and non-reducing conditions and blotted with monoclonal antibody (mAb) 16H3 (1 μg/ml) and goat-anti-mouse IgG (whole molecules, Sigma, St. Louis). Fourteen μl of culture supernatant from HIV-1 Env-transfected DHFR-deficient CHO cells were used. AE.A244 HIV-1 wild type (WT) env-transfected CHO cells, B.63521WT env-transfected CHO cells, and B.6240 WT env-transfected CHO cells were harvested at the second round of gene amplification with 100 nM of MTX and C.1086 WT env-transfected cells were harvested at the third round of gene amplification with 200 nM of MTX. DHFR-deficient CHO cells grew in adapted serum-free media. B.63521 WT env-transfected and B.6240 WT env-transfected CHO cells produced cleaved gp120 envelope proteins while AE.A244WT env-transfected and C.1086 WT env-transfected CHO cells generated non-cleaved forms of HIV-1 gp120 proteins.

FIGS. 2A and 2B. HIV-1 Env proteins expressed in HEK293 cells by transient transfection. For transient expression of HIV-1 glycoproteins, plasmids expressing HIV-1 gp120 Env proteins were transfected into HEK293 cells and cell lysates were prepared. HTV-1 env genes were subcloned into the pcDNA3.1(+)/Hygro eukaryotic expression vector (Invitrogen, Carlsbad, Calif.) via a XbaI site and a BamHI site under the control of the CMV immediate early promoter. Two mg of purified plasmid was transfected into HEK293 cells (ATCC, Bethesda, Md.) at approximately 80% confluence with polyethylenime (MW 25,000, Cat #23966, Polysciences Inc., Warrington, Pa.) per the manufacturer's protocol, Recombinant HIV-1 proteins were purified using lectin agarose beads (Galanthus Nivalis, Vector Laboratories, Burlingame, Calif.). The purified Env proteins were fractionated on 4-12% gradient SDS-PAGE under reducing and non-reducing conditions. Shown are Western blots under reducing and non-reducing conditions with 0.25 μg of purified Env protein per lane (FIG. 2A)—1 μg of purified protein was loaded per lane in Coomassie staining (FIG. 2B). HIV-1 gp120 proteins expressed in HEK293 cells by transient transfection were not cleaved (FIGS. 2A and 2B), Fractionated HIV-1 Env proteins were detected by mAb 16H3 (1 μg/ml) and goat-anti-mouse IgG.

FIG. 3. Western blot analysis of HIV-1 B.63521 gp140WT expressed in HEK293 cells and DHFR-deficient CHO cells. B.63521 gp140WT proteins expressed in HEK293 cells via transient transfection produced an intact form of HIV-1 gp140 protein 3 days after small scale transfection. However, in DHFR-deficient CHO cells, B.63521 gp140WT generated cleaved forms of HIV-1 gp140 at the second round of gene amplification with 100 nM of MTX in adapted serum-free medium, The HIV-1 gp140 proteins were fractionated on 4-12% gradient SDS-PAGE under reducing and non-reducing conditions and blotted with mAb 16H3 and goat-anti-mouse IgG. Fourteen al of culture supernatant were used per lane from DHFR-deficient CHO cell lines.

FIG. 4. Repair of the cleavage sites by substitution of V3 sequences in mutant Env proteins. B.63521 gp120 wild type proteins and cleavage-repaired mutant gp120 proteins were purified using lectin agarose beads from DHFR-deficient CHO cell cultures. The purified envelope proteins were fractionated on 4-12% gradient SDS-PAGE under non-reducing and reducing conditions. 0.25 μg of purified Env protein was loaded per lane in Western blot analysis while 1 μg of Env proteins was loaded in Coomassie staining. HIV-1 gp120 proteins expressed in the stably transfected DHFR-deficient CHO cells were harvested at the second round of gene amplification with 100 nM MTX. Culture supernatants of B.6240 gp120 wild type-transfected and cleavage-repaired B.6240 gp120 mutant-transfected CHO cells were analyzed by Western blot and harvested at the first round of gene amplification with 50 nM MTX. Fractionated HIV-1 Env proteins were detected by mAb 16H3 and goat-anti-mouse IgG.

### DETAILED DESCRIPTION OF THE INVENTION

The present invention relates, at least in part, to a method of eliminating cleavage of recombinant HIV-1 Envs produced, for example, in DHFR-deficient CHO cells. As detailed in the Example that follows, most of HIV-1 gp120 proteins expressed in CHO cells are cleaved, while the same gp120 proteins expressed in HEK293 (293F) cells are produced as intact proteins. Similarly, HIV-1 B.63521 gp140 Env proteins are produced as cleaved forms in CHO cells, while the same gp140 proteins express as intact proteins in HEK293 cells. In SDS-PAGE, the cleaved HIV-1 Env proteins produced in CHO cells appear as intact proteins under non-reducing conditions, however, they migrate as ˜75Kd and ˜50Kd cleaved proteins bands under reducing conditions. These results suggest that HIV-1 Env gp120 and gp140 proteins are produced as cleaved products and appear as intact proteins as a result of disulfide bond formation. In contrast, C.1086 gp120 proteins are expressed as intact proteins in both HEK293 and CHO cells.

It has been reported that HIV gp120 is frequently cleaved during expression as a recombinant protein at a single site in the V3 loop between R³¹⁵ and A³¹⁶. Other reports suggest that the cleavage site is in a type II β-turn centered at P³¹³-G³¹⁴ (Du et al, Protein Expr. Purif. 59:223-231 (2008), Niwa et al, Eur. J. Biochem. 237:64-70 (1996)). HIV-1 B.9021 Env, which has a gp120 R³¹⁵ and T³¹⁶ sequence in the V3 loop, has also been found to be frequently cleaved when expressed in CHO cells.

The study described in the Example that follows was undertaken to determine if the V3 loop sequence of the C.1086 gp120 protein (TRPNNNTRKSIRIGPGQTFYATGDIIGNIRQAH) could be used as a template for modifying other HIV-1 gp 120 isolates so as to render them resistant to cleavage when produced in CHO cells. The results provided in the Example demonstrate that substitution of amino acid residues in the crown of the V3 loop of B.63521 gp120, B.6240 gp120, and B.9021 gp140 with amino acids corresponding to those in the crown of the C.1086 gp120 isolates renders them resistant to cleavage when produced in CHO cells.

Accordingly, the invention relates to a method producing an HIV-1 Env protein (gp120 or gp140, or subunit thereof) that is resistant to cleavage when produced in CHO cells (e.g., DHFR-deficient CHO cells) comprising substituting amino acid residues in the V3 loop (e.g,, the crown of the V3 loop) of such a protein to produce a protein that has the V3 loop sequence TRPNNNTRKSIRIGPGQTFYATGDIIGNIRQAH. The substitution can be effected using methods well known in the art, including site-directed mutagenesis. The invention relates to the mutant proteins and to nucleic acids comprising a nucleotide sequence encoding same. The invention further relates to methods of producing the mutant proteins recombinantly comprising introducing into a host cell, e.g., a DHFR-deficient CHO cell, a nucleic acid construct comprising a nucleotide sequence encoding the mutant protein and culturing the resulting host cell under conditions such that the mutant protein is produced. The mutant protein can be isolated from the host cells using methods known in the art.

Certain aspects of the invention can be described in greater detail in the non-limiting Example that follows.

### EXAMPLE

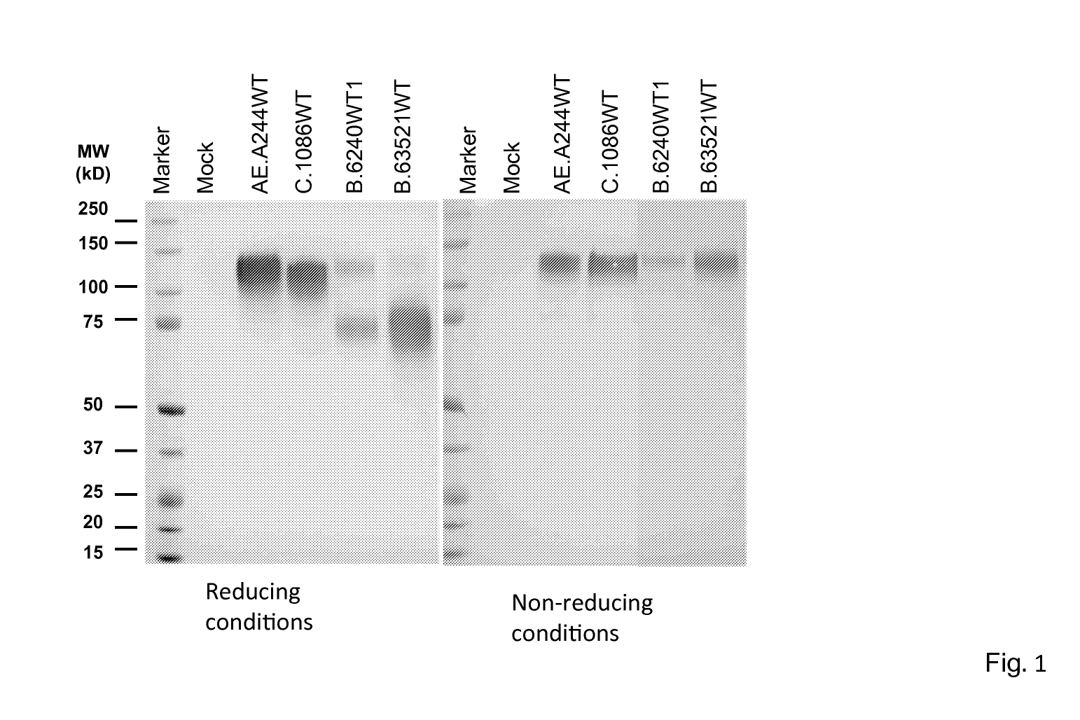

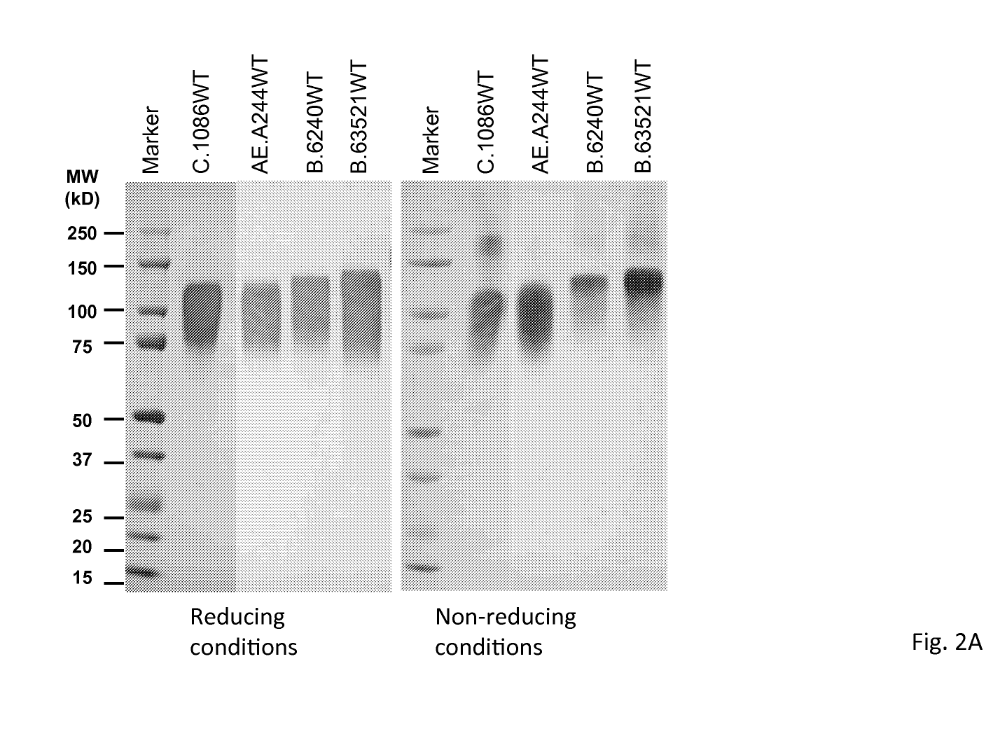

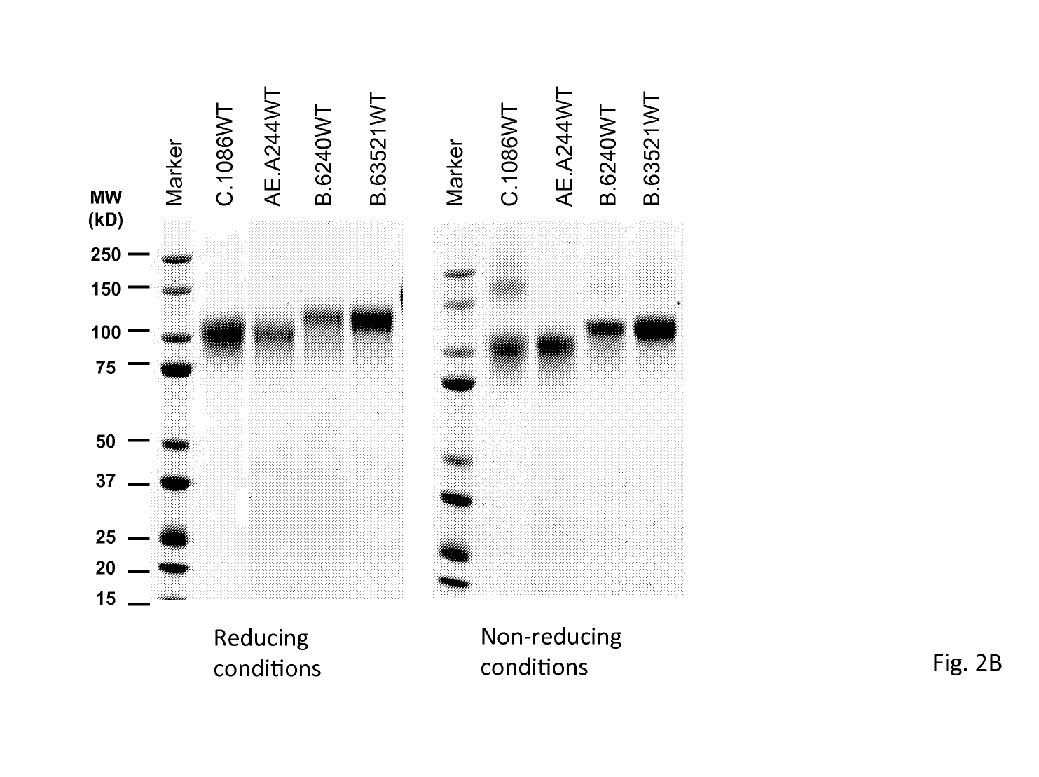

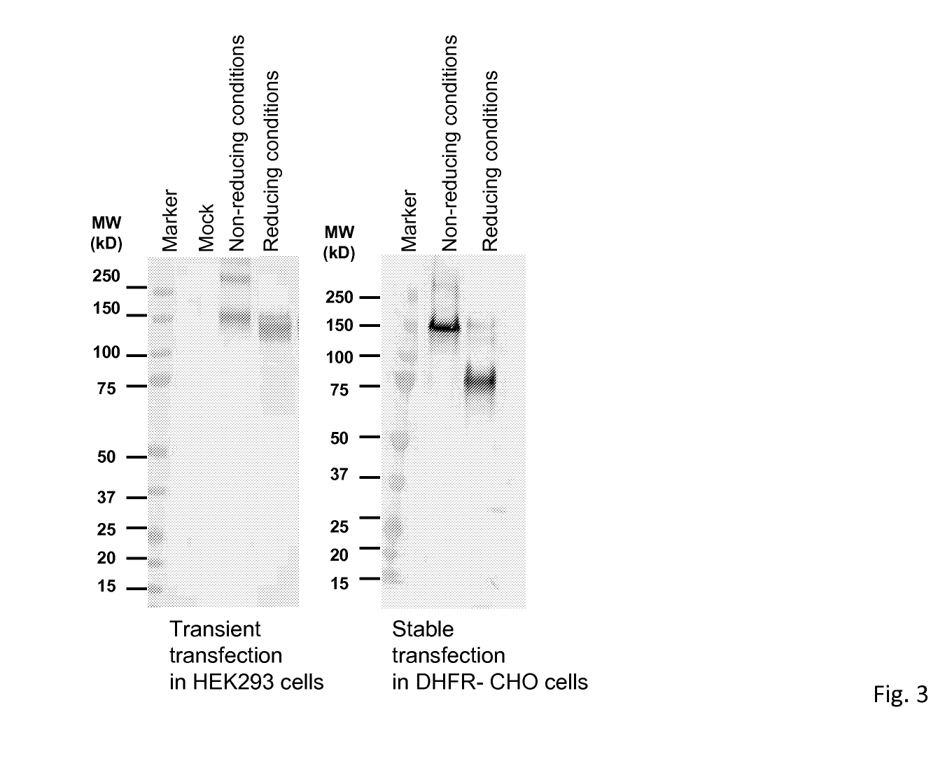

In an effort to establish stably transfected CHO cell lines, it was found that most of HIV-1 gp120 proteins expressed in CHO cells were cleaved (FIG. 1), while the same gp120 proteins expressed in HEK293 (293F) cells were produced as intact proteins (FIG. 2). Similarly, HIV-1 B.63521 gp140 Env proteins were generated as cleaved forms in CHO cells, while the same gp140 proteins expressed as intact proteins in HEK293 cells (FIG. 3). In SDS-PAGE, the cleaved HIV-1 Env proteins produced in CHO cells appeared as intact proteins under non-reducing conditions but migrated as ˜75 Kd and ˜50 Kd cleaved proteins bands under reducing conditions in FIG. 1. These results suggested that HIV-1 Env gp120 and gp140 proteins were produced as cleaved products and appeared as intact proteins as a result of disulfide bond formation holding cleaved protein fragments together. In contrast, C.1086 gp120 proteins were expressed as intact proteins in both HEK293 and CHO cells (FIGS. 1 and 2).

It has been reported that HIV gp120 is frequently cleaved during expression as a recombinant protein at a single site in the V3 loop between R³¹⁵ and A³¹⁶ by thrombin. Other observations also suggest that the cleavage site is in a type II β-turn centered at P³¹³-G³¹⁴ (Du et al, Protein Expr. Purif. 59:223-231 (2008), Niwa et al, Eur. J. Biochem. 237:64-70 (1996)). However, HIV-1 B.9021 Env that has a gp120 R³¹⁵ and T³¹⁶ sequences in the V3 loop also was found to have >90% of protein expressed as the cleavage product when expressed in CHO cells,

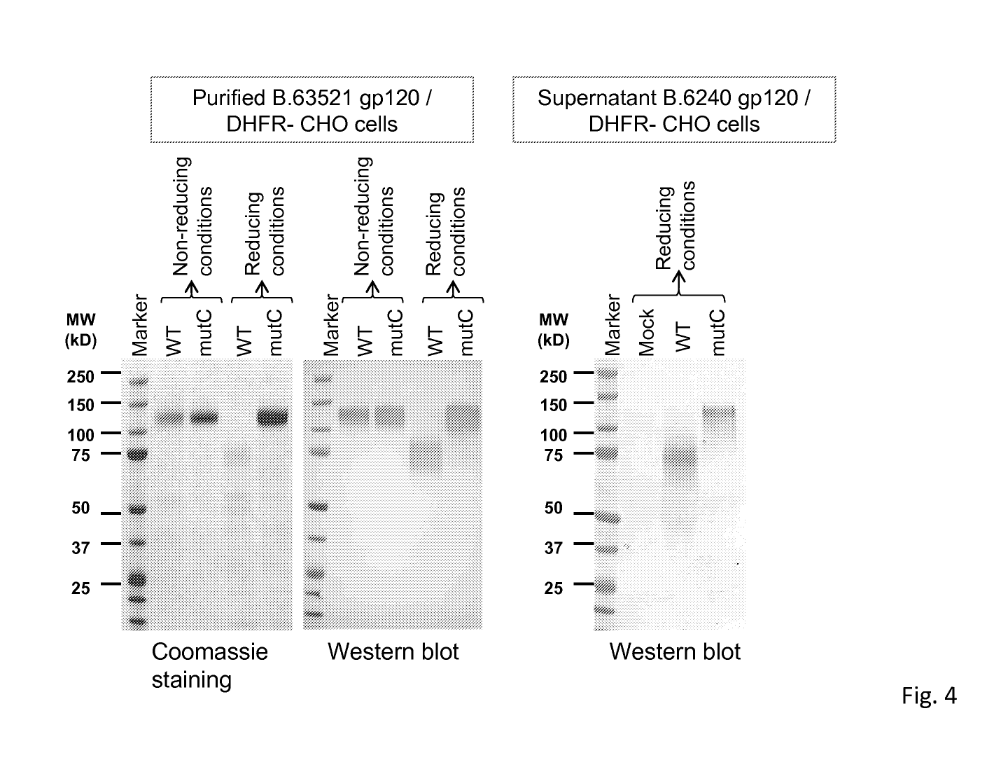

To determine if the V3 loop sequence of the C.1086 gp120 protein could be used as a template for modifying the sequence in other HIV-1 gp 120 isolates so that they were protected from cleavage when expressed in CHO cells, amino acid residues in the crown of the V3 loop of B.63521 gp120, B.6240 gp120, and B.9021 gp140 were substituted with amino acids from the crown of the C.1086 gp120 by site-directed mutagenesis resulting in B.63521 gp120mutC, B.6240 gp120mutC and B.9021 gp140mutC Env sequences (Table 1). B.63521 gp120mutC, B.6240 gp120mutC, and B.9021 gp140mutC plasmids were produced and used to generate stably transfected CHO cell lines. When produced in stably transfected CHO cell lines, purified B.63521 gp120mutC proteins, as well as 13.6240 gp120mutC and B.9021 gp140mutC proteins, appeared uncleaved in SDS-PAGE under non-reducing and reducing conditions (FIG. 4).

Amino acid residues of wild type and mutant HIV-1
envelopes

| HIV-1 Env ID | V3 loop sequence |
|---|---|
| C.1086WT | TRPNNNTRKSIRIGPGQTFYATGDIIGNIRQAH |
| B.63521WT | TRPNNNTRKSIHIGPGRAFYATGETIGNIRQAH |
| B.63521mutC | TRPNNNTRKSIRIGPGQTFYATGEIIGNIRQAH |
| B.6240WT | TRPNNNTRKGIHIGLGRALYATGDIIGDIRQAH |
| B.6240mutC | TRPNNNTRKSIRIGPGQTFYATGDIIGDIRQAH |
| B.9021WT | TRPGNNTRKSIHIAPGRTFYATGEHGDIRRAH |
| B.9021mutC | TRPNNNTRKSMIGPGQTFYATGETIGDIRRAH |

It has been known that the V3 loop is a major neutralization target against HIV-1, is involved in co-receptor binding, and plays a role in cell tropism (Hartley et al, AIDS Res. Hum. Retroviruses 21:171-189 (2005)). Therefore, minor changes of amino acids residues in V3 loop could alter the antigenicity of HIV-1 Env proteins. The binding reactivities of wild type and mutant gp140/gp120 proteins produced in HEK293 cells to a panel of 14 monoclonal antibodies (mAbs) were evaluated by an ELISA (Table 2). mAbs in this panel include 17B, 19B, 2G12, 697D, A32, CH31, B12, PG9, CH01, and 2F5. Overall, HIV-1 gp120WT and gp120mutC Envs showed very similar reactivity against various HIV-1 mAbs in ELISA assay (Table 2). There were no differences in the binding reactivity between B.63521 WT and B/63521mutC Envs, while B.6240mutC showed slightly better binding data (EC₅₀) than B.6240WT such as 0.004 vs>100 to mAb 19B and 0.218 vs 1.370 to B12 (Table 2). In the case of B.9021 gp140B.9012 gp140WT showed similar or better binding reactivity than B.9021 gp140mutC. Interestingly, B.9021 gp140 mutC loses its binding activity to Cat_CH01 such as 0.577 (B.9021 gp140WT) vs 14.490 (B.9021 gp140mutC). Cat_CH01 is a broadly neutralizing mAb that could react to V2/V3 conformational epitope of HIV-1 envelope (Bonsignori et al, J. Virol. 86:4688-4692 (2011)). B.9021 gp140mutC loses its reactivity to mAb PG9 that reacted to V1V2 conformational epitope, 3.209 (B.9021 gp140WT) vs 11.345 (B.9021 gp140mutC). Thus, the biological characteristics of these mutant HIV-1 gp120 Envs are nearly identical to their wild type proteins with regard to reactivity of the Env antibodies tested.

TABLE 2
Binding of HIV-1 monoclonal antibodies to wild type and mutant envelopes^{a}

| | EC₅₀ (μM) | | | | | |
|---|---|---|---|---|---|---|
| | Envelope | | | | | |
| | B.63521 | B.63521 | B.6240 | B.6240 | B.9021 | B.9021 |
| Antibody | WT | mutC | WT | mutC | gp140WT | gp140mutC |
| 17B CL2 | 0.122 | 0.212 | 0.036 | 0.041 | 0.024 | 0.358 |
| 19B | 0.006 | 0.005 | >100 | 0.004 | 0.004 | 0.003 |
| Cat_2G12_AAA | 0.013 | 0.013 | 0.010 | 0.009 | 0.008 | 0.011 |
| Cat_697_ AAA | 0.007 | 0.008 | 0.008 | 0.007 | 0.016 | 0.228 |
| Cat_A32_ AAA | 0.029 | 0.027 | 0.023 | 0.016 | 0.060 | 0.660 |
| Cat_CH31 | 0.536 | 0.617 | 1.323 | 0.738 | 1.793 | 6.999 |
| IgG1 B12 | 0.100 | 0.109 | 1.370 | 0.218 | 0.110 | 0.434 |
| PG9 | 0.121 | 0.133 | 0.066 | 0.033 | 3.209 | 11.345 |
| 2F5 | — | — | — | — | 0.116 | 0.053 |
| Cat_CH01 | — | — | — | — | 0.577 | 14.490 |

^{a}The table shows 50% effective concentration (EC50) values determined from direct-binding ELISAs.
“—” indicates negative results, as defined by OD 450 readings below twice the background at the highest antibody concentration tested.

All documents and other information sources cited herein are hereby incorporated in their entirety by reference.

What is claimed is:

1. A method of producing an HIV-1 Env protein that is resistant to cleavage when produced in CHO cells comprising substituting amino acid residues in the V3 loop of an HIV-1 Env protein that is cleaved when produced in CHO cells to yield a protein that has the V3 loop sequence TRPNNNTRKSIRIGPGQTFYATGDIIGNIRQAH.
